(19) United States
(12) Patent Application Publication
(10) Pub. No.: US 2007/0105768 A1
(43) Pub. Date: May 10, 2007

(54) Dry recombinant human alpha 1-antitrypsin formulation

(76) Inventors: Rajiv Nayar, Pleasant Hill, CA (US); Mark C. Manning, Loveland, CO (US); Philip J. Barr, Oakland, CA (US); Philip A. Pemberton, San Mateo, CA (US); Ian C. Bathurst, Alameda, CA (US); Helen Gibson, Oakland, CA (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/578,692

(22) PCT Filed: Nov. 10, 2004
(86) PCT No.: PCT/GB04/04740

§ 371 Date: Aug. 26, 2006

(51) Int. Cl. A61K 38/36

(52) U.S. Cl. 514/12

(57) ABSTRACT

A dry powder composition comprises recombinant human alpha 1-antitrypsin (rAAAT).

### FIELD OF THE INVENTION

This invention relates to a dry protein formulation, and in particular to a formulation of alpha 1-antitrypsin (AAT).

### BACKGROUND OF THE INVENTION

AAT and recombinant alpha 1-antitrypsin (rAAT) are potential therapeutic agents for a number of clinical indications. rAAT is a 395 amino acid protein of 44 kD, that is non-glycosylated and has an amino acid sequence identical to the human plasma protein (AAT) with the exception of an N-acetylmethionine residue at the amino terminus. It is desirable to have a dry, stable formulation of AAT or rAAT, ready for reconstitution in water and immediate use.

Excipients typically employed in a dried protein formulation (see, for example, Carpenter et al, Pharm. Biotechnol. 13:109-133,2002) comprise mainly buffers, sugars and surfactants. Other potential stabilizing excipients include bulking agents, chelating agents, antioxidants, reducing agents and amino-acids.

U.S. Pat. No. 5,780,014A describes a dry powder formulation of AAT, for administration by inhalation. Various drying techniques are suggested.

Prolastin (Bayer) is a lyophilized preparation of human plasma-derived, glycosylated AAT. When reconstituted as directed, at 1 g alpha 1-antitrypsin functional activity per 40 mL sterile water, the liquid composition comprises >20 mg/ml AAT, 100-210 mEq/L Na, 60-1 80 mEq/L Cl, 15-25 μM sodium phosphate, <5 ppm PEG and <0.1% sucrose. The lyophilized formulation should be stored under refrigeration.

Vemuri et al, in Chapter 9 of Stability and Characterization of Protein and Peptide Drugs: Case Histories, ed. Wang and Pearlman, Plenum Press, New York (1993); describe formulations of rAAT, primarily in liquid form. Stability, e.g. at pH 7.5, is enhanced by increasing the salt content. However, salt is generally considered unsuitable for a lyophilized formulation because of the reduced glass transition temperature. The stabilization of. rAAT presents particular problems, relative to the natural protein. Travis et al., (J. Biol. Chem. 260:4384-4389,1985) describe a comparison of heat stabilities of yeast-derived rAAT with natural plasma-derived AAT. The half-life of non-glycosylated rAAT, with respect to its activity in response to thermal stress, is considerably less than that of its natural glycosylated counterpart.

### SUMMARY OF THE INVENTION

The present invention is based on the discovery of a dry formulation of rAAT, having defined concentrations of rAAT and salt, that has good stability, even without refrigeration (i.e. at 5″C or below), of up to 2 years or more. This can be achieved without losing other desirable properties such as rapid reconstitution and a clear resultant solution. The content of excipients, especially any that could potentially promote microbial growth, can be minimized, and non-approved or non-compendial chemicals can be avoided. The formulation has no offensive odour or taste. It is amenable to a convenient lyophilization cycle.

### BRIEF DESCRIPTION OF THE DRAWINGS

FIG. 1 is a FTIR spectral scan of liquid and solid rAAT in a formulation of the invention.

FIG. 2 is a FTIR spectral scan of unformulated rAAT in the liquid and solid states.

FIG. 3 is a FTIR spectral scan of rAAT in formulations containing different levels of salt.

FIG. 4 shows the secondary structure of rAAT in a sugar-based formulation and in a salt-based formulation of the invention.

FIG. 5 illustrates the reversibility of the secondary structure of rAAT to its original structure upon reconstitution of the lyophilized protein in 100 mM NaCl formulation.

### DESCRIPTION OF PREFERRED EMBODIMENTS

A dried formulation according to the invention contains at least rAAT and salt. Although their effect on the stability of the composition is relatively small, other, conventional components may be included. Such components include reducing agents such as dithiothreitol, cysteine, glutathione, or N-acetylcysteine (NAC), e.g. in an amount of up to 10 mM on reconstitution. The composition may also contain antioxidants, such as ascorbic acid or L-Met, e.g. in an amount of up to 10 mM on reconstitution and/or a buffer such as phosphate, citrate or histidine, e.g. in an amount of 5-50 mM, preferably 10-20 mM on reconstitution. The amount of buffer may be such that, on reconstruction of the composition in water, the reconstituted solution has a pH of from about 6 to 9, more preferably 6.5-8, preferably from 6.8-70.

Other typical constituents are chelating agents (e.g. EDTA or citrate), and surfactants (e.g., polyoxyethylene sorbitan). These and any other components may be present in any combination. These additional components are optional, and it is preferred that the novel formulation contains as few of these additional components as necessary.

The dry powder composition of the invention does not require to have been subjected to viral inactivation. That is typically done by heating, at 60° C. or 65° C.

The dry powder composition of the invention typically has a protein content which is less than 10%, more preferably less than 5%, most preferably less than 1%, α1-antichymotrypsin. The composition also typically has protein content which is less than 10%, more preferably less than 5%, most preferably less than 1%, albumin. More generally, protein content is usually less than 10%, more preferably less than 5%, most preferably less than 1% human protein. The protein content is usually more than 90%, preferably more than 95% rAAT, and most preferably more than 99% rAAT.

The dry powder composition may further comprise 1 to 2000 milliequivalents salt per 100 mg of rAAT, more preferably 50-500 milliequivalents, most preferably 100-200 milliequivalents. The salt that is used will typically be NaCl. However, it will be readily appreciated by those of ordinary skill in the art that other salts may have the same effect, whether the cation is different (as in KCl) or the anion is different (as in NaBr) or both.

The dry powder composition of the invention can be free of sugar. It usually contains less than 1% and preferably less than 0.5% water.

The dry powder composition of the invention can retain at least 80% of initial rAAT activity, preferably >90%, upon storage at under conditions that are, or are equivalent to, 50° C. for 3 months. The composition may also retain at least 80% monomeric rAAT, preferably >95% monomer, upon storage under conditions that are, or are equivalent to, 50° C. for 3 months.

Criteria for stability (retained activity) and denaturation are demonstrated by assays known to those skilled in the art. Activity assays are based on the porcine pancreatic elastase inhibition assay reported by Beatty et al, J Biol. Chem. 255, p. 3931, 1980. Denaturation is monitored by evaluation of aggregate formation, and the non-denatured rAAT reported as % monomer, in a size exclusion chromatography (SEC) HPLC method. Equivalence to the given conditions will be understood by one of ordinary skill in the art, i.e. based on the Arrhenius equation.

In order to prepare a formulation of the invention, a solution or other composition comprising the desired components is dried. Suitable methods of drying include, but are not limited to, lyophilization, spray-drying, spray freeze-drying, fluidized bed technology and super critical fluid drying.

Preferred drying procedures are lyophilization and spray-drying. Both procedures can be performed by standard technology known to those of ordinary skill in the art. For example, spray-drying consists of a three-step process which results in dry particle formation. The process begins by atomizing a liquid feed into a spray of fine droplets using compressed air, followed by heating media in order to dry the droplets by evaporating the moisture content of the droplets. The final particles in the form of dry powder are collected as product. The gas and the excess fine dust are exhausted. These steps are carried out using three components: the atomizer in shape of a nozzle; the drying chamber; and the collecting system known as cyclone and pot.

The dry formulation or, after reconstitution, the liquid composition is suitable for administration to a patient in need thereof. Suitable routes of administrations include, but are not limited to, inhalation, topical, sub-cutaneous and intravenous delivery.

The following Examples illustrate the invention.

The following abbreviations (not already explained before) are used:

NaPi: sodium phosphate

Tw8O: Tween 80 (Tween may be a registered Trademark)

FTIR: Fourier transform infrared

### EXAMPLE 1

### Lyophilization

The formulations shown in Table I were made.

TABLE I

| | [AAT] | | | | | | | |
|---|---|---|---|---|---|---|---|---|
| Sample | mg/ml | pH | NaPi | Histidine | NaCl | Citrate | NAC | L-m |
| 917-1 | 50 | 7 | 20 | 0 | 175 | 5 | 2.5 | 3 |
| 917-3 | 50 | 7 | 20 | 0 | 100 | 5 | 2.5 | 3 |
| 917-4 | 50 | 7 | 20 | 0 | 50 | 5 | 2.5 | 3 |
| 917-11 | 50 | 7 | 20 | 0 | 0 | 0 | 0 | 0 |

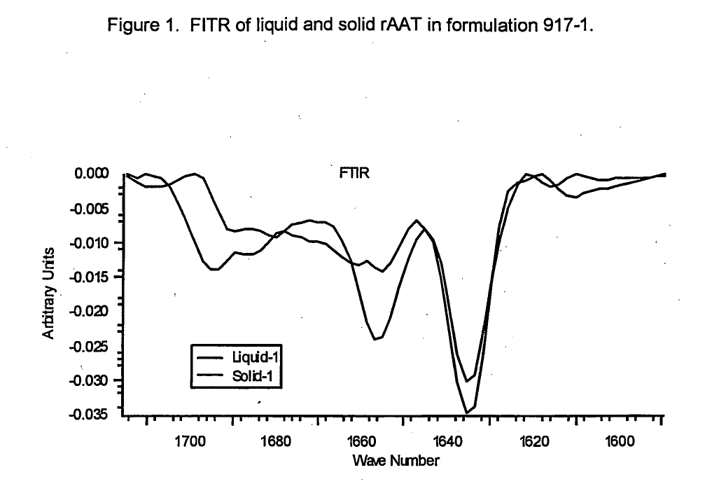

In order to assess the conformational stability of rAAT in the dried state, FTIR spectra were collected on these formulations. It has been shown that retention of native structure in the solid state can be predictive of long-term storage stability for dried proteins (Carpenter et al, 2002, supra). FIG. 1 shows the FTIR of liquid and solid rAAT in Formulation 917-1. Note that the amide I region (1700-1600 cm⁻¹) is sensitive to changes in secondary structure and that all peaks in the second derivative spectra are negative. Each peak in the amide I region corresponds to a different secondary structural type. There are clearly perturbations of the rAAT conformation before and after lyophilization. The peak near 1655 cm⁻¹ corresponds to α-helical structure, the band near 1635 cm⁻¹ corresponds to β-sheet structure, and the 1688 cm⁻¹ peak arises from extended β-strands or β-sheets. Random coil structure is assigned to bands near 1644 cm⁻¹.

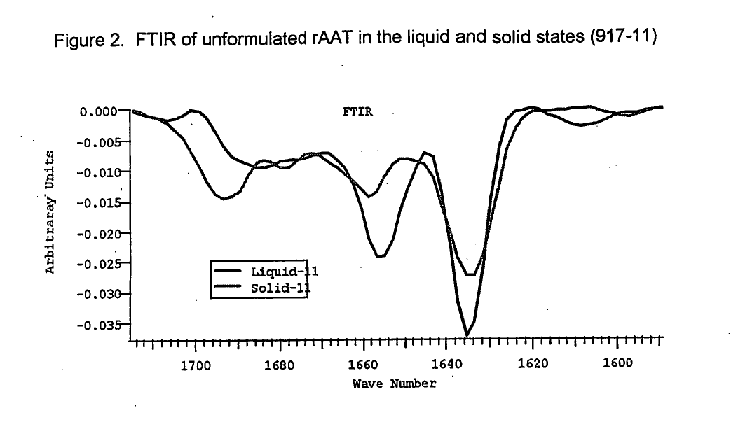

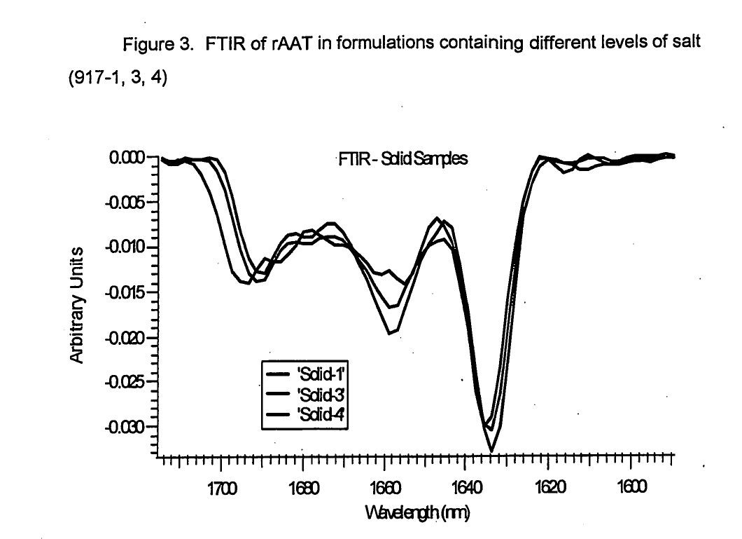

The liquid sample, representing the native conformation, displays a significant amount of β-sheet and α-helical structure. Upon lyophilization, without stabilizers (formulation 917-11), there is significant structural perturbation as shown in FIG. 2. The α-helix band is almost completely lost, while there are marked increases in bands above 1680 cm⁻¹, corresponding to extended and loop structures. FIG. 3 shows the effect of salt on rAAT structure in the solid state. Formulations 917-1, -3 and -4 contain 175 mM, 100 mM and 50 mM NaCl, respectively, in addition to 20 mM sodium phosphate, 5 mM citrate, 2.5 mM NAC, and 3 mM L-Met.

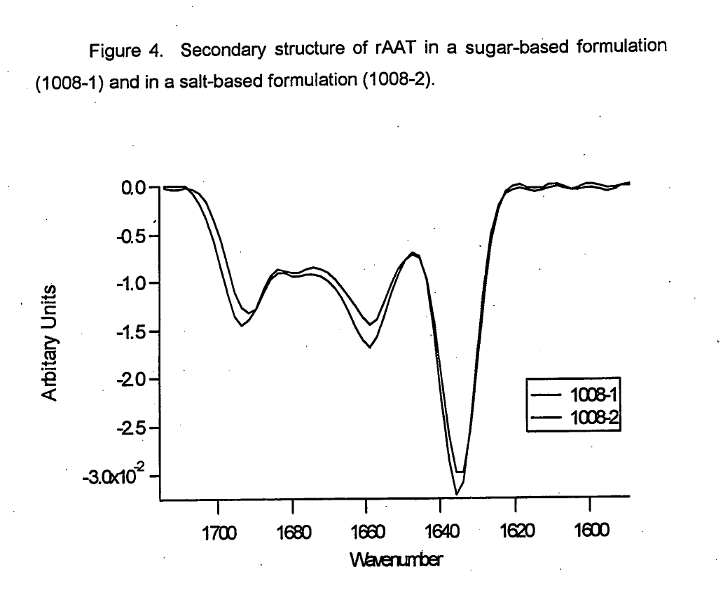

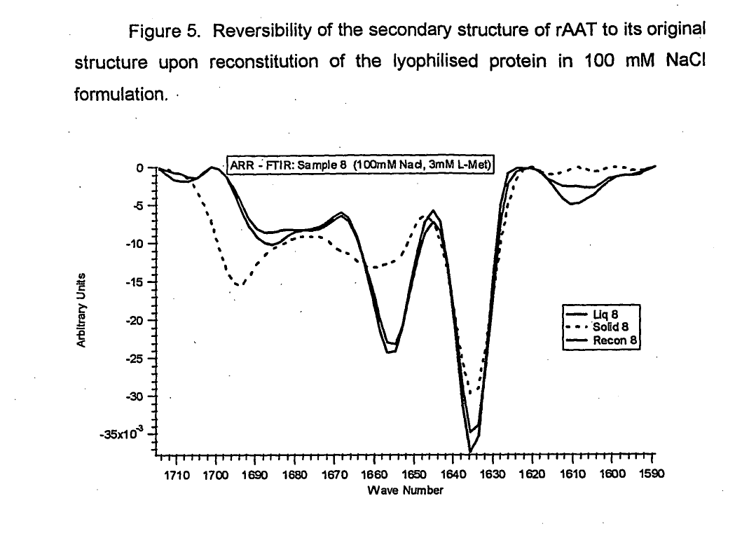

Formulations 3 and 4, which have the lower salt concentrations, appear to have the greatest degree of structural perturbation and all three formulations are less perturbed than when no stabilizers are present. Overall, it appears that lyophilization produces some structural perturbation compared to the native conformation. The extent of the changes is minimized by the addition of excipients, including salt. It appears that a NaCl concentration above 50 mM produces a more native-like structure, with a 50-100 mM optimum. The result is unanticipated, since sugars are usually required or used to maintain native protein structure in the dried state. Conformational stability of these formulations was also assessed by FTIR in order to elucidate any subtle differences between the rAAT structure in the dried state. FIG. 4 shows the FTIR spectra of rAAT formulated in a sugar-based formulation (1008-1) and in a salt-based formulation (1008-2). The secondary structure of rAAT in both these formulations is superimposable. The fact that salt can accomplish the same degree of stabilization with protein at high concentrations is remarkable and not obvious. Upon reconstitution, the original rAAT secondary structure is retained as shown in FIG. 5.

Based on the surprising observations of lyophilized rAAT formulations containing high levels of NaCl, stability analysis were done in order to evaluate systematically whether addition of common stabilizers in lyophilized protein formulations enhances the conformational stability and acute stability (3 month storage at 60° C.) of rAAT. Sugars are commonly used in protein formulations to stabilise the molecule by presumably substituting for the H-bonding following removal of the water molecules around the protein during lyophilization. Sugars also offer an amorphous environment in the dry state that promotes conformational stability of the protein, and they effectively replace the water of hydration removed during drying. Surfactants are also often employed in protein formulations to reduce surface adsorption that may damage the protein. Since a possible administration route for rAAT is pulmonary delivery via aerosolization, the effect of surfactant is especially of interest. Therefore, the role of polyoxyethylene sorbitan, such as polysorbate 80 (Tween 80), at various concentrations was also evaluated. These formulations are given in Table II.

TABLE II

| | | NaPi | Trehalose | Sucrose | Tw80 | NaCl | L-met | NAC | Citrate |
|---|---|---|---|---|---|---|---|---|---|
| Sample | pH | mM | % | % | % | mM | mM | mM | mM |
| 1008-1 | 7.4 | 10 | 5 | 0 | 0 | 0 | 5 | 0 | 0 |
| 1008-2 | 6.8 | 10 | 0 | 0 | 0 | 100 | 3 | 0 | 0 |
| 1008-3 | 6.8 | 10 | 0 | 0 | 0 | 100 | 3 | 0 | 0 |
| 1008-4 | 6.8 | 10 | 2.5 | 0 | 0 | 100 | 3 | 0 | 0 |
| 1008-5 | 6.8 | 10 | 2.5 | 0 | 0 | 100 | 3 | 0 | 0 |
| 1008-6 | 6.8 | 10 | 2.5 | 0 | 0 | 100 | 3 | 0 | 0 |
| 1008-7 | 6.8 | 10 | 0 | 2.5 | 0 | 100 | 3 | 0 | 0 |
| 1008-8 | 7.4 | 10 | 0 | 0 | 0 | 100 | 3 | 0 | 0 |
| 1008-9 | 6.8 | 10 | 0 | 0 | 0 | 100 | 3 | 2.5 | 5 |
| 1008-10 | 6.8 | 10 | 0 | 1 | 0 | 100 | 3 | 0 | 0 |
| 1008-11 | 6.8 | 10 | 0 | 2.5 | 0 | 100 | 3 | 0 | 0 |
| 1008-12 | 6.8 | 10 | 0 | 5 | 0 | 100 | 3 | 0 | 0 |

The lyophilized formulations were evaluated for short-term stability (at 1 and 3 months) under accelerated storage conditions at 60° C. It should be noted that this storage temperature is particularly harsh for evaluating protein stability and may bias the results towards the trehalose-based formulations that have a particularly high glass-transition temperature (Tg). The rationale for choosing this temperature was based on previous stability studies that assessed rAAT stability over shorter time frames. The activity and percent monomer recovered were determined for up to 3 months storage at 60∞ C., as shown in Tables III and IV, respectively.

TABLE III
Specific Activity of rAAT (IU/mg)

| | | Lyo | | Lyo | Lyo |
|---|---|---|---|---|---|
| | Pre- | (1 month | | (1 month | (3 months |
| Sample | lyo | RT) | Moisture | 60° C.) | 60° C.) |
| liquid | 3.75 | | | | |
| control | | | | | |
| 1008-1 | 3.6 | 3.45 | 0.4% | 3.42 | 2.4 |
| 1008-2 | 3.69 | 2.83 | 1.4% | 2.89 | 2 |
| 1008-3 | 4.02 | 3.22 | | 3.21 | 2 |
| 1008-4 | 3.57 | 3.47 | 0.6% | 3.31 | 2.6 |
| 1008-5 | 3.67 | 3.21 | | 3.24 | 2.7 |
| 1008-6 | 3.7 | 3.10 | | 3.43 | 2.5 |
| 1008-7 | 3.89 | 3.08 | | 3.22 | 2.6 |
| 1008-8 | 3.43 | 3.15 | | 3.09 | 2.3 |
| 1008-9 | 3.57 | 3.16 | | 3.23 | 2.5 |
| 1008-10 | 3.38 | 3.16 | | 3.12 | 2.7 |
| 1008-11 | 4.04 | 3.28 | 0.4% | 3.2 | 2.7 |
| 1008-12 | 3.31 | 3.31 | | 3.18 | 3 |

TABLE IV
Percent Monomer by Size Exclusion HPLC

| | | Lyo | Lyo | Lyo |
|---|---|---|---|---|
| Sample | Pre-lyo | (1 month RT) | (1 month 60° C.) | (3 month 60° C.) |
| liquid | 97.6 | | | |
| control | | | | |
| 1008-1 | 98.2 | 97.02 | 96.7 | 74.71 |
| 1008-2 | 97.4 | 95.65 | 96.7 | 65.57 |
| 1008-3 | 97.4 | 96.03 | 94.89 | 60.62 |
| 1008-4 | 97.4 | 96.04 | 96.3 | 85.85 |
| 1008-5 | 97.4 | 96.36 | 96.07 | 86.94 |
| 1008-6 | 97.3 | 96.73 | 96.16 | 83.2 |
| 1008-7 | 97.4 | 96.33 | 95.29 | 86.76 |
| 1008-8 | 97.5 | 96.3 | 94.26 | 65.46 |
| 1008-9 | 97.1 | 96.03 | 93.12 | 74.32 |
| 1008-10 | 97.2 | 96.3 | 94.35 | 84.29 |
| 1008-11 | 97.3 | 95.72 | 95.03 | 82.83 |
| 1008-12 | 97.2 | 96.19 | 95.96 | 5.41 |

No significant differences were observed in any of the formulations tested after 1 month, suggesting that both sugar-containing and sugar-free formulations offer comparable stability.

The stability data after storage for 3 months at 60° C. display more variable results. It appears that formulations containing both sugar and salt have a better stability profile than those containing either sugar or salt alone. These data are consistent with FTIR studies that show a high degree of retention of secondary structure in these types of formulations. The low specific activity seen in formulation 1008-2 may be due to the moisture content in that formulation, which is almost 1% higher than that determined in the other selected formulations. This suggests that stable lyophilized rAAT formulations should preferably have a moisture content below 1%.

These results suggest that rAAT is a relatively stable protein and may not require sugars for stabilization in the lyophilized state.

### EXAMPLE 2

### Spray Drying

Recombinant alpha 1-antitrypsin (rAAT) was spray-dried in various formulations and conditions. The activity of the resulting dry powder was assayed to evaluate the rAAT potency after drying. Table VII presents the formulations and Table VIII presents the data from these experiments.

TABLE VII

| | rAAT | | NaPi | NaCl | NAC | Citrate | L-Met |
|---|---|---|---|---|---|---|---|
| Formulation | (mg/ml) | pH | mm | mm | mm | mm | mm |
| ARV-8 | 10 | 6.8 | 10 | 100 | 0 | 0 | 3 |
| ARV-9 | 10 | 6.8 | 10 | 100 | 2.5 | 5 | 3 |
| ARV-13 | 10 | 6.8 | 10 | 100 | 5 | 1 | 0 |

TABLE VIII

| | Spray Dry Temp | |
|---|---|---|
| | Conditions Inlet/Outlet | Specific Activity |
| Formulation | ° C./° C. | U/mg |
| AVR-8 Retain | | 3.3 |
| ARV-8 | 110/77 | 2.2 |
| ARV-8 | 80/63 | 2.9 |
| AVR-9 Retain | | 4.5 |
| ARV-9 | 80/62 | 4 |
| ARV-9 | 110/77 | 4.4 |
| AVR-13 Retain | | 4.9 |
| ARV-13 | 80/62 | 2.9 |
| ARV-13 | 110/77 | 4.3 |

The features described above are not exhaustive. Other embodiments are within the scope of the invention.

All references cited herein are incorporated by reference.

1. A dry powder composition comprising recombinant human alpha 1-antitrypsin (rAAT).

2. The dry powder composition of claim 1, that has not been subjected to viral inactivation.

3. The dry powder composition of claim 1, whose protein content is less than 10% α1-antichymotrypsin.

4. The dry powder composition of claim 1, whose protein content is less than 10% albumin.

5. The dry powder composition of claim 1, whose protein content is less than 10% human protein.

6. The dry powder composition of claim 1, whose protein content is more than 90% rAAT.

7. The dry powder composition of claim 6, whose protein content is more than 95% rAAT.

8. The dry powder composition of claim 6, whose protein content is more than 99% rAAT.

9. The dry powder composition of claim 1, further comprising 1 to 2000 milliequivalents salt per 100 mg of rAAT.

10. The dry powder composition of claim 1, that is free of sugar.

11. The dry powder composition of claim 1, that contains less than 1% water.

12. The dry powder composition of claim 11, that contains less than 0.5% water.

13. The dry powder composition of claim 1, that retains at least 80% of initial rAAT activity upon storage under conditions that are, or are equivalent to, 50° C. for 3 months.

14. The dry powder composition of claim 1, that retains at least 80% monomeric rAAT upon storage under conditions that are, or are equivalent to, 50° C. for 3 months.

15. The dry powder composition of claim 1, further comprising a reducing agent.

16. The dry powder composition of claim 1, further comprising an antioxidant.

17. The dry powder composition of claim 1, further comprising a buffer.

18. The dry powder composition of claim 17, wherein the buffer is such that, on reconstitution of the composition in water, the reconstituted solution has a pH of from about 6 to 9.

19. The dry powder composition of claim 1, further comprising a chelating agent.

20. The dry powder composition of claim 1, further comprising a surfactant.

21. The dry powder composition of claim 1, that consists essentially only of rAAT and 1 to 2000 millequivalents salt per 100 mg of rAAT, a reducing agent, an antioxidant, a buffer and a surfactant.
